(19) United States
(12) Patent Application Publication
(10) Pub. No.: US 2022/0152121 A1
(43) Pub. Date: May 19, 2022

(54) Composition and Use of Interleukin Stimulated Human Umbilical Cord Mesenchymal Stem Cells for the Treatment of Rheumatoid Arthritis

(71) Applicant: National Yang Ming Chiao Tung University, Hsinchu (TW)

(72) Inventors: Hwai-Shi WANG, Hsinchu (TW); Jeng-Jong HWANG, Hsinchu (TW)

(21) Appl. No.: 17/342,158

(22) Filed: Jun. 8, 2021

(30) Foreign Application Priority Data
Nov. 18, 2020 (TW) 109140255

(51) Int. Cl. A61K 35/51; A61P 19/02; C12N 5/0775

(52) U.S. Cl. CPC A61K 35/51; C12N 2501/2301; C12N 5/0665; A61P 19/02

(57) ABSTRACT

The present invention relates to a composition and method for treating rheumatoid arthritis, comprising interleukins (ILs), including IL-1β, stimulated human umbilical cord mesenchymal stem cells (hUCMSCs). The IL stimulated hUCMSCs of the present invention may induce the apoptosis of fibroblast-like synoviocytes rheumatoid arthritis (HFLS-RA) cells. IL stimulated hUCMSCs are useful to alleviate RA symptoms, such as inflammation, swelling and cartilage erosion, and exhibit therapeutic effects on rheumatoid arthritis.

### BACKGROUND OF THE INVENTION

### Technical Field of the Invention

The present invention relates to a pharmaceutical composition for treating rheumatoid arthritis and method thereof by using human umbilical cord mesenchymal stem cells produced by interleukin pre-stimulation. More specifically, it relates to the use of a pharmaceutical composition comprising human umbilical cord mesenchymal stem cells pre-stimulated by interleukin-1β for the treatment of rheumatoid arthritis.

### Background

Rheumatoid arthritis (RA) is an autoimmune joint disease characterized by synovial proliferation and lymphocyte accumulation leading to progressive damage of the periarticular and articular structure. The synovium consists of fibroblast-like synoviocytes (FLSs) and macrophage-like synoviocytes (MLSs), with FLSs being the predominant cell type in the synovial intima. The hyperplasia of the synovium lining and pannus mainly consists of tissue-invading FLSs, infiltrating lymphocytes and macrophages. The increasing proliferation and/or insufficient apoptosis might contribute to the expansion of RA FLSs, suggesting that the inducing of apoptosis in RA, FLSs could be a therapeutic approach (Pope, R. M. Nature Reviews Immunology. 2:527, 2002). Therefore, the induction of FLSs apoptosis is one potential treatment strategy for rheumatoid arthritis.

Human umbilical cord mesenchymal stem cells (hUCMSCs) have self-renewal and multipotent differentiation properties. Mesenchymal stem cells (MSCs) also exert paracrine effects and are capable of migrating to inflammation sites. MSCs promote angiogenesis, enhance tissue regeneration in injured sites and exhibit anti-inflammatory and immunomodulatory effects. The therapeutic potential of bone marrow-derived mesenchymal stem cells (BM-MSCs) has investigated in an RA animal model. The results show that BM-MSCs provide conditionally therapeutic benefits (Papadopoulou et al., Annals of the rheumatic diseases: annrheumdis-2011-200985, 2012). Intra-articular knee implantation of autologous BM-MSCs in phase ½ clinical trial reveals that it is safe and well tolerated for further investigation (Shadmanfar et al., Cytotherapy, 20:499-506, 2018). Treatment of anti-rheumatic drugs with hUCMSCs has been found to enhance the therapeutic efficacy for patients with active RA (Wang et al., Stem cells and development, 22:3192-3202, 2013).

In an inflammatory environment, the induced proliferation of FLSs by some inflammatory factors (such as TGF-β, TNF-α, IL-10) can be inhibited by hUCMSCs (Liu, Y. et al., Arthritis research & therapy. 12:R210, 2010). The elevated proliferation of FLSs and the insufficient apoptosis of the cells are key factors causing rheumatoid arthritis. Therefore, inducing the apoptosis of human fibroblast like synoviocyte-rheumatoid arthritis (HFLS-RA) to reduce the damage of surrounding bones and articular cartilage is one treatment strategy. hUCMSCs are easier to obtain than bone marrow mesenchymal stem cells, however, there are few studies that focus on their application in animal models of rheumatoid arthritis.

The present invention first discloses that in cell experiments, IL-1β enhances the expression of TRAIL in hUCMSCs and the expression of DR4 and DR5 in HFLS-RA, thereby inducing apoptosis of HFLS-RA and inhibiting abnormal proliferation of synoviocytes. In live animal experiments, hUCMSCs stimulated by IL-1β protected the normal function of joints by significantly reducing the inflammation and swelling as well as the erosion of articular cartilage by synoviocytes caused by rheumatoid arthritis.

### SUMMARY OF INVENTION

Accordingly, in one aspect, the present invention relates to a pharmaceutical composition for the treatment of rheumatoid arthritis, comprising human umbilical cord mesenchymal stem cells (hUCMSCs) pre-stimulated by interleukin and pharmaceutically acceptable carriers, diluents or excipients. In some embodiments, the hUCMSCs are pre-stimulated with 5-1000 ng/ml (preferably 50-200 ng/ml) of interleukin for 8-72 hours, preferably 16-48 hours, and most preferably 24-30 hours. In a preferred embodiment, the said interleukin is interleukin-1β (IL-1β).

In some embodiments, the hUCMSCs pre-stimulated with interleukin is used to promote the apoptosis of HFLS-RA. In some embodiments, the hUCMSCs pre-stimulated with interleukin is used to inhibit the abnormal proliferation of synoviocytes, thereby preventing the erosion of articular cartilage by synoviocytes. In other embodiments, the hUCMSCs pre-stimulated with interleukin is used to relieve the inflammation and swelling of rheumatoid arthritis.

In one aspect, the present invention relates to a use of hUCMSCs pre-stimulated by interleukin for the treatment of rheumatoid arthritis, comprising said hUCMSCs pre-stimulated with 5-1000 ng/ml (preferably 50-200 ng/ml) of interleukin for 8-72 hours, preferably 16-48 hours, and most preferably 24-30 hours. In a preferred embodiment, the said interleukin is IL-1β.

In some embodiments, the hUCMSCs pre-stimulated with interleukin is used to inhibit the proliferation of fibroblast-like synoviocytes. In some embodiments, the hUCMSCs pre-stimulated with interleukin is used to reduce joint inflammation and swelling. In some embodiments, the hUCMSCs pre-stimulated with interleukin is used to inhibit the erosion of articular cartilage in patients with arthritis.

In one aspect, the present invention relates to a method for treating rheumatoid arthritis, comprising administration of hUCMSCs pre-stimulated with interleukin to a patient in need. In some embodiments, the hUCMSCs is pre-stimulated with 5-1000 ng/ml (preferably 50-200 ng/ml) of interleukin for 8-72 hours, preferably 16-48 hours, and most preferably 24-30 hours. In a preferred embodiment, the said interleukin is IL-1β.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1E depict the effect of IL-1β stimulation on ICAM-1 expression in HFLS-RA cells: FIG. 1A shows HFLS-RA cells were stimulated with 50, 100, 200 ng/ml IL-1β for 24 hours. The expression of ICAM-1 was examined by Western blot. FIG. 1B shows quantitative results of the Western blot ICAM-1 expression of FIG. 1A (n=6). FIG. 1C shows the images of immunofluorescence showed ICAM-1 expression in different concentrations of IL-1β stimulation for 24 hours. Green: ICAM-1, blue: Hoechst 33258 (nucleus), scale bar: 100 μm. FIG. 1D shows quantitative fluorescence intensity results of FIG. 1C analyzed by image J. FIG. 1E shows immunofluorescence images captured by using laser confocal microscope. Scale bar: 10 μm.

FIGS. 2A-2B depict the effect of IL-1β on cell adhesion ability using fluorescence study. FIG. 2A shows hUCMSCs and HFLS-RA cells were pre-stimulated with or without 100 ng/ml IL-1β for 24 hours, then co-cultured for an hour. hUCMSCs were labeled with Calcein AM, and nuclei were stained with Hoechst 33258. FIG. 2B shows quantitative the cell number of five random field of view normalized to hUCMSCs without IL-1β stimulation, the results were analyzed by image J. The data represent mean±SD (*P<0.05, **P<0.01, ***P<0.001).

FIGS. 3A-3E depict the expressions of TRAIL in hUCMSCs stimulated with different IL-1β concentrations. FIG. 3A shows hUCMSCs were stimulated with 50, 100, 200 ng/ml IL-1β for 24 hours. The expression of TRAIL was examined by Western blot. FIG. 3B shows Western blot of TRAIL expression in FIG. 3A was quantified (n=6). FIG. 3C shows immunofluorescent images of TRAIL expressions under different concentrations of IL-1β stimulation for 24 hours. Green: TRAIL, blue: Hoechst 33258 (nucleus), scale bar: 100 μm. FIG. 3D shows quantitative results of FIG. 3C analyzed by image J (n=5). FIG. 3E shows immunofluorescent images of TRAIL expression captured by laser confocal microscope. Scale bar: 10 μm. The data represent mean±SD (*P<0.05, **P<0.01, ***P<0.001).

FIGS. 4A-4D depict the effect of IL-1β stimulation on DR4 and DR5 expression in HFLS-RA cells. FIG. 4A shows HFLS-RA cells were stimulated with 50, 100, 200 ng/ml IL-1β for 24 hours. The expression of DR4 was examined by Western blot. FIG. 4B shows quantitative results of the Western blot DR4 expression of FIG. 4A (n=7). FIG. 4C shows the expression of DR5 was examined by Western blot. FIG. 4D shows quantitative results of the Western blot DR5 expression of FIG. 4C (n=5) (*P<0.05).

FIGS. 5A-5F depict immunofluorescence study of DR4 and DR5 protein expression with different IL-1β concentration in HFLS-RA cells. FIG. 5A shows HFLS-RA cells were stimulated with 50, 100, 200 ng/ml IL-1β for 24 hours. The expression of DR4 was examined by immunofluorescent staining. Green: DR4, blue: Hoechst 33258 (nucleus), scale bar: 10 μm. FIG. 5B shows the expression of DR5 was examined by immunofluorescent staining. Green: DR5, blue: Hoechst 33258 (nucleus), scale bar: 10 μm. FIG. 5C shows immunofluorescence study of DR4 expression with 100 ng/ml IL-1β stimulation in 6, 16, 24, 48 hours. Green: DR4, blue: Hoechst 33258 (nucleus), scale bar: 100 μm. FIG. 5D shows Quantitative DR4 fluorescence intensity results of FIG. 5C analyzed by image J. (n=4). FIG. 5E shows immunofluorescence study of DR5 expression with 100 ng/ml IL-1β stimulation in 6, 16, 24, 48 hours. Green: DR5, blue: Hoechst 33258 (nucleus), scale bar: 100 μm. FIG. 5F shows quantitative DR5 fluorescence intensity results of FIG. 5E analyzed by image J. (n=3). The data represent mean±SD (*P<0.05, **P<0.01, ***P<0.001).

FIGS. 6A-6C depict the ability of hUCMSCs inducing apoptosis of HFLS-RA cells. FIG. 6A shows after 24 hours of co-culture, cells were stained with Annexin V/PI detection kit. hUCSMCs were labelled with CellTracker Orange. Annexin V represented as early apoptosis, and PI represented as late apoptosis. Green: Annexin V, red: nucleus—PI, whole cell with light red hUCMSCs, scale bar: 100 μm. FIG. 6B shows the expression of caspase 3 after co-culturing with hUCMSCs by immunofluorescence study. Green: caspase 3, blue: Hoechst 33258 (nucleus), scale bar: 100 μm. FIG. 6C shows the expression of caspase 8 after co-cultured with hUCMSCs by immunofluorescence study. Green: caspase 8, blue: Hoechst 33258 (nucleus), scale bar: 100 μm.

FIGS. 7A-7B depict the therapeutic efficacy evaluation of hUCMSCs in a CIA mouse model. FIG. 7A shows statistical results of arthritis score after hUCMSCs administration. FIG. 7B shows statistical results of paw thickness after hUCMSCs administration. The data represent mean±SD (*P<0.05, **P<0.01, ***P<0.001).

FIG. 8 depicts the therapeutic efficacy of hUCMSCs evaluated by H&E stain. CIA mice were sacrificed on day 40 after hUCMSCs administration. The joint tissues were collected and pathological examination of synovial hyperplasia and bone destruction was performed. Scale bar: 500 μm. White arrow head: synovial hyperplasia. Red arrow head: the distance of the joint cavity.

FIG. 9. The inflammation of arthritis was assayed by micro-positron emission tomography/computerized tomography ([¹⁸F]FDG/microPET-CT).

FIG. 10 depicts the therapeutic efficacy of hUCMSCs in RA-bearing mice by evaluating the exterior appearance after hUCMSCs administration. Mice were anesthetized to record the exterior appearance in order to evaluate therapeutic efficacy. On the day 40, with hUCMSCs and IL-1β-hUCMSCs administration, the symptoms of redness and swelling were significantly improved. Arrow head: the redness and swelling of the front and hind paws.

### DETAILED DESCRIPTION OF THE INVENTION

Other features and advantages of the present invention are further exemplified and described in the following examples, which are intended to be illustrative only and not to limit the scope of the invention.

### Example 1, IL-1β Promotes the Adhesion of hUCMSCs to HFLS-RA

Human umbilical cord mesenchymal stein cells (hUCMSCs) were maintained in low serum defined medium, which consisted of 56% low-glucose Dulbecco's Modified Eagle Medium (DMEM-LG; Invitrogen, CA, USA), 37% MCBD 201 (Sigma, MO, USA), 2% fetal bovine serum (Thermo, Logan, Utah), 0.5 mg/ml of AlbuMAX® I (Life Technologies, NY, USA), 1× Insulin-Transferrin-Selenium-A (Life Technologies, NY, USA), 10 nM dexamethasone (Sigma, MO, USA), 10 ng/ml epidermal growth factor (PeproTech, NJ, USA), 50 nM L-ascorbic acid 2-phosphate (Sigma, MO, USA), and 1 ng/ml of platelet-derived growth factor-BB (PeproTech, NJ, USA). The cells were incubated in a humidified incubator with 5% CO2 at 37° C.

Before cytokine stimulation, hUCMSCs were starved in serum-free DMEM-LG medium for 16 hours. Then cells were treated with 5-1000 ng/ml (for example, 50-200 ng/ml) interleukin 1β (IL-1β) for 24-30 hours to give IL-1β-stimulated hUCMSCs.

IL-1β Stimulates ICAM-1 Expression in HFLS-RA Cells

ICAM-1 participates in many immunological response processes, including adhesion and transendothelial migration of immune cells to inflammation sites. As a marker of HFLS cells, ICAM-1 expression with IL-1β was investigated to mimic the inflammation environment. After stimulation with 5-1000 ng/ml (for example, 50-200 ng/ml) IL-1β for 24-30 hours, the expression of ICAM-1 was evaluated by Western blot.

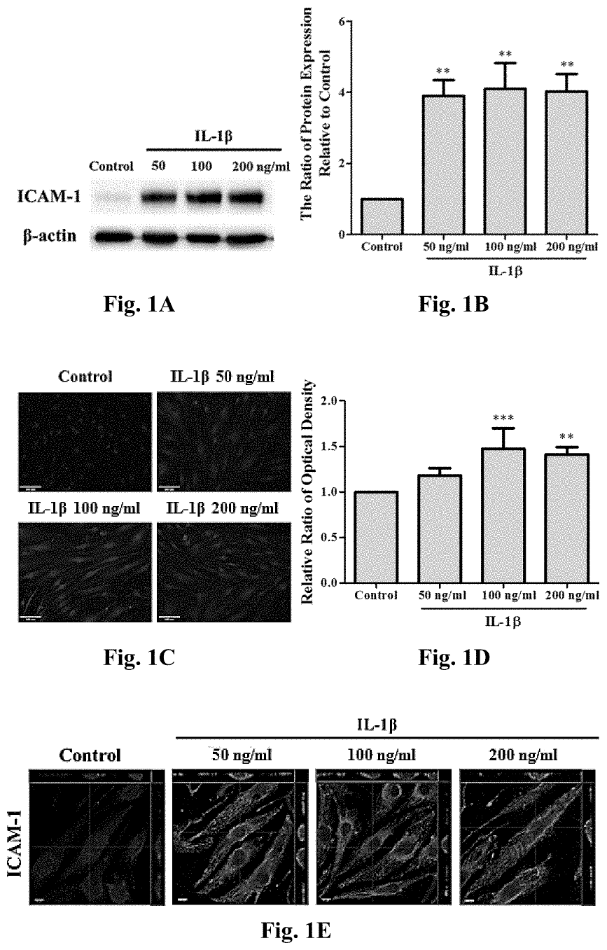

The results of Western blot showed that ICAM-1 was significantly increased (FIG. 1A), and had the highest expression at the concentration of 100 ng/ml IL-1β (FIGS. 1B and 1D). Since ICAM-1 is a ligand of LFA-1, the protein distribution on cell surfaces were also examined by capturing the images using a laser confocal microscope. The results showed that ICAM-1 expression was increased on cell surfaces after IL-1β stimulation (FIG. 1E).

The Effect of IP-1β in Cell Adhesion Ability

hUCMSCs and HTLS-RA cells were stimulated with 100 ng/ml IL-1β for 24 hours, respectively. hUCMSCs (5×10⁴/900 μl) were labeled with 6 μM Calcein AM (Tocris, UK), then transferred into each well which containing HELS-RA, and incubated for an hour at 37° C. in the dark. Then, cells were stained with Hoechst 33258 (Sigma, MO, USA) and mounted with fluorescence mounting medium (Ibidi, Planegg, Germany). Cells were imaged using a fluorescent microscope (Leica. DM600013, Wetzlar, Germany), and cell number was counted under five random fields of view (FOV).

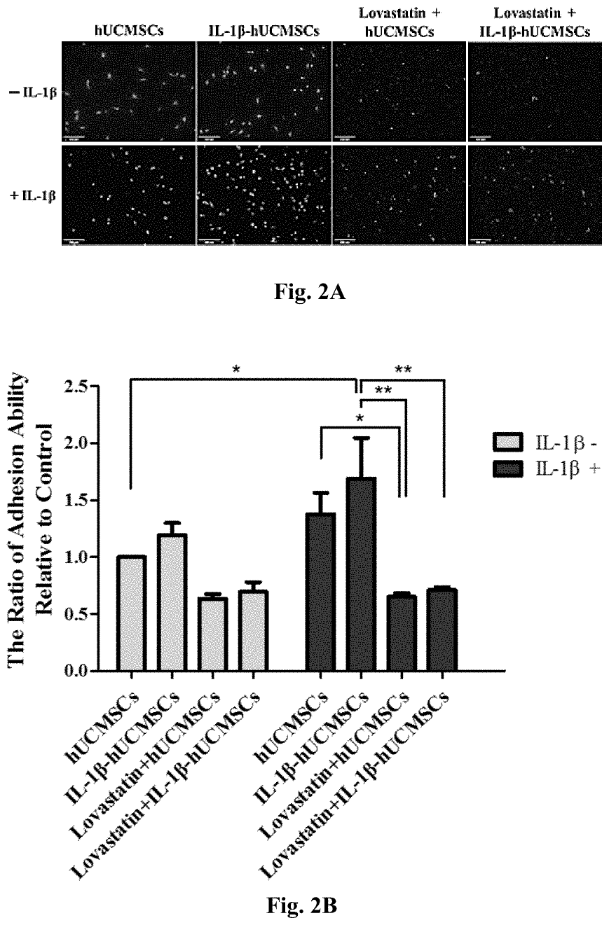

In this adhesion assay, the result showed that the adhering number of hUCMSCs pre-stimulated with IL-1β was significantly increased compared with unstimulated cells (FIG. 2). However, the number of adhering cells were significantly decreased when pre-treated with Lovastatin, an LFA-1 antagonist, with or without IL-1β. These results suggest that HFLS-RA cells and hUCMSCs may interact via ICAM-1/LFA-1 interaction.

IL-1β Stimulates TRAIL Expression in hUCMSCs

Tumor necrosis factor (TNF)-related apoptosis-inducing ligand (TRAIL or Apo 2 ligand), which belongs to the tumor necrosis factor (TNF) superfamily, is a type II transmembrane protein, Recently, TRAIL has been applied in cancer therapy research due to its apoptosis-inducing potential. There are five TRAIL receptors: death receptor 4 (DR4, TRAIL-R1), DR5 (TRAIL-R2), decoy receptor 1 (DcR1, TRAIL-R3). DcR2 (TRAIL-R4), and osteoprotegrin (OPG). TRAIL could interact with DR4 and DR5, which contains the death domain, and induce the cell apoptosis.

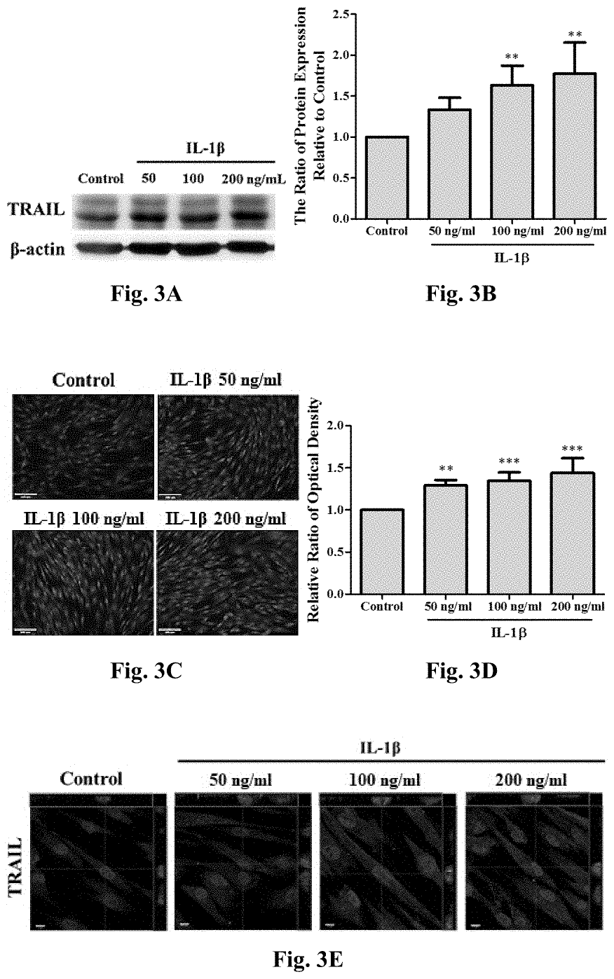

To evaluate the effect of TRAIL expression in hUCMSCs after stimulation with 5-1000 ng/ml (for example, 50-200 ng/ml) IL-1β for 24-30 hours, Western blot and immunofluorescence were performed. The results showed that the expression of TRAIL increased after IL-1β stimulation and had a dose-dependent effect (FIGS. 3A-3D). As TRAIL is a member of type II transmembrane protein, the functional protein expressed on cell surfaces were evaluated using a laser confocal microscope to capture the images. The results showed that TRAIL was increased on the cell surface after IL-1β stimulation (FIG. 3E).

IL-1β Stimulates DR4 and DR5 Expression in HFLS-RA Cells

HFLS-RA cells have been found to express TRAIL receptors DR4 and DR5. Highly expressed IL-1β is found in the inflamed joint tissues of patients with rheumatoid arthritis. In order to further confirm whether IL-1β stimulation affects the protein expression of apoptosis receptors DR4 and DR5 in a pathological environment, The HFLS-RA cells were stimulated with IL-1β at a concentration of 5-1000 ng/ml (for example, 50-200 ng/ml) for 24-30 hours, and the expression of DR4 and DR5 was examined.

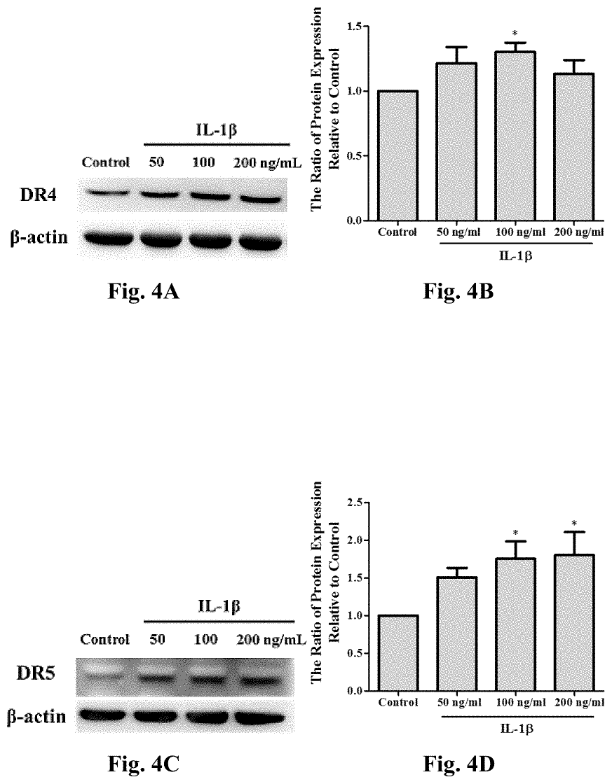

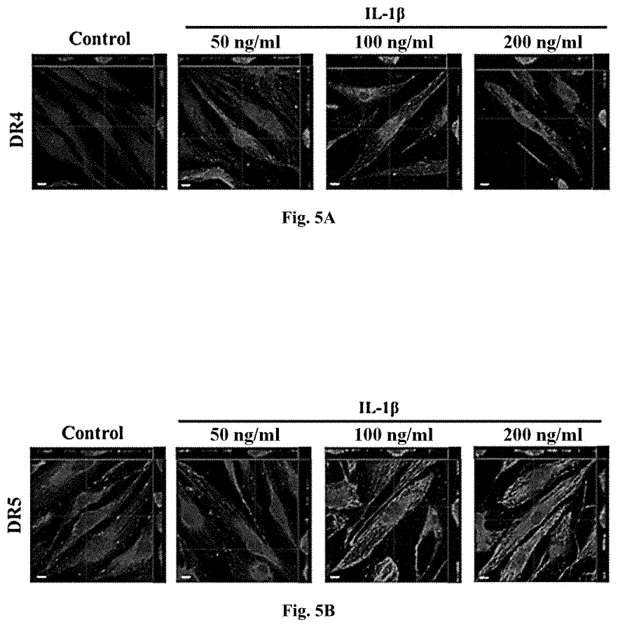

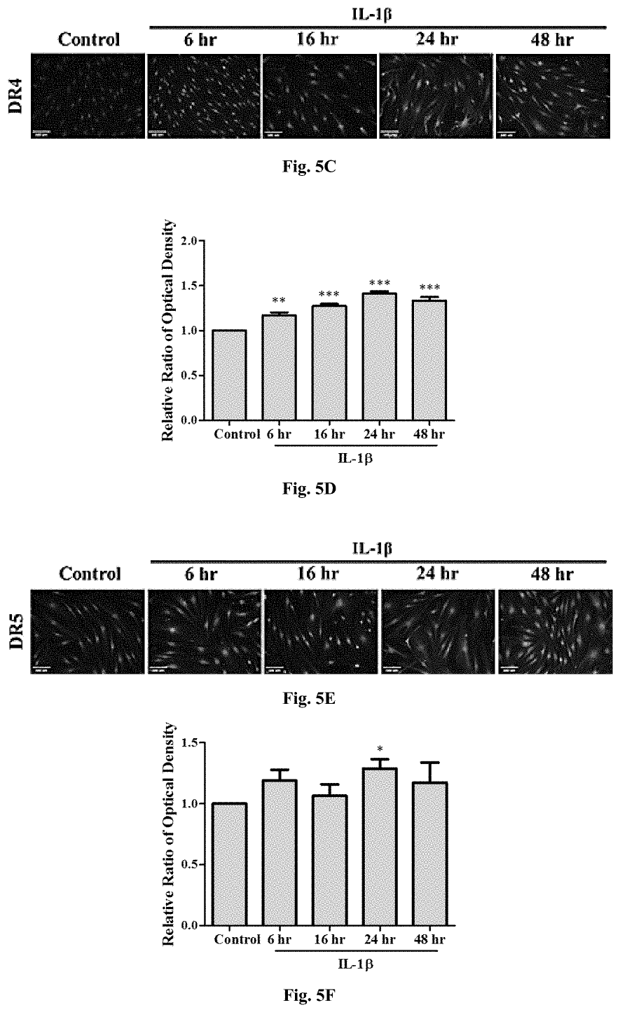

The results of Western blot showed that both DR4 and DR5 were upregulated after IL-1β stimulation. At the concentration of 100 ng/ml IL-1β, DR4 had the highest expression (FIGS. 4A and 4B). DR5 exhibited dose-dependent effects with IL-1β stimulation (FIGS. 4C and 4D). DR4 and DR5 expressions were also confirmed by immunocytostaining. The results showed that expressions of DR4 (FIGS. 5A and 5C) and DR5 (FIGS. 5B and 5E) were significantly upregulated with IL-1β stimulation and reached the highest levels at 24 hours, respectively (FIGS. 5D and 5F).

Based on the results of this example, it is shown that after IL-1β stimulation, the expression of TRAIL of hUCMSCs and the expression of ICAM-1 of HFLS-RA cells are increased, thereby increasing the ability of hUCMSCs to adhere to HFLS-RA cells. IL-1β can also increase the expressions of DR4 and DR5 on the surface of HFLS-RA cells.

### Example 2. IL-1β-Stimulated hUCMSCs Induces Apoptosis of HELS-RA Eats after Direct Co-Culturing

HFLS-RA cells were seeded on 12 mm microscope coverslips and cultured in 24-wells plate, and hUCMSCs were cultured in 10 cm dish. Both hUCMSCs and HFLS-RA cells were stimulated with 100 ng/ml IL-1β for 24 hours, respectively. After IL-1β stimulation, hUCMSCs were labelled with 5 μM CellTracker Orange (Life Technology, NY, USA) for 30 minutes at 37° C., protected from light. After labelling, 5×10⁴ hUCMSCs were added to 24-wells plate which contained HFLS-RA cells. After co-culturing for 24 hours, cells were stained with Annexin V-FITC Apoptosis Detection Kit (Strong Biotech Corporation, Taipei, Taiwan). After incubation, samples were immediately examined and the images were captured using a fluorescent microscope (Leica DM6000B, Wetzlar, Germany). hUCSMCs were labelled with CellTracker Orange. Annexin V represented as early apoptosis, and PI represented as late apoptosis.

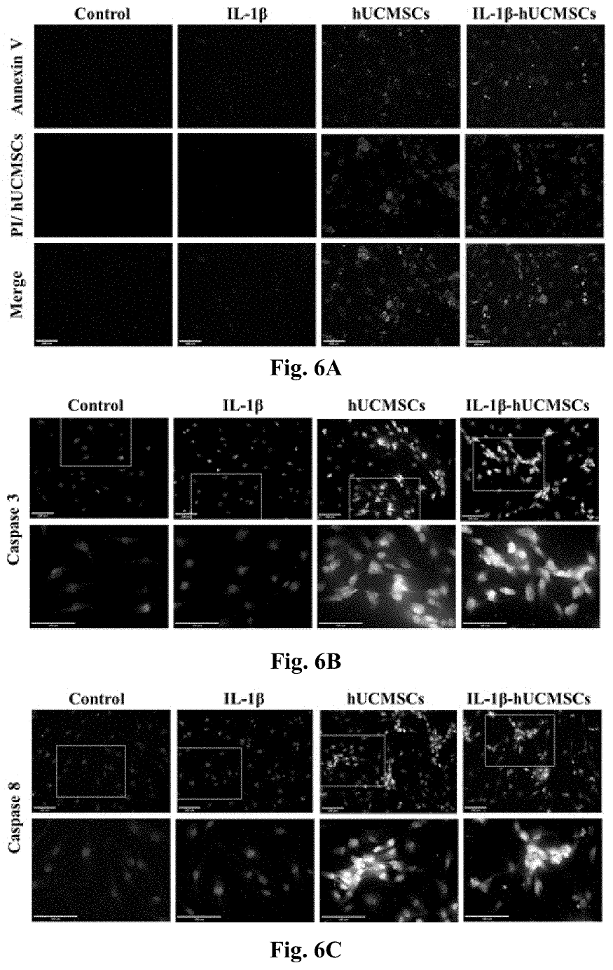

The results of immunofluorescent staining (FIGS. 6A-6C) showed that both caspases 8 and 3 were upregulated with hUCMSCs co-culturing. Caspases 8 and 3 were highly expressed in HFLS-RA cells which were around hUCMSCs. It indicated that hUCMSCs induced the apoptosis of HFLS-RA cells via cell-cell contact.

According to the cell experiment results of the above examples 1 and 2, IL-1β stimulated hUCMSCs adhering to FLSs occurred via LFA-1/ICAM-1 interaction while apoptosis was induced via TRAIL/DR4, DR5 interaction.

### Example 3, Therapeutic Efficacy Evaluation of hUCMSCs Administration in CIA Mouse Model

Animal experimental protocols were approved by the Institutional Animal Care and Use Committee (IACUC) of National Yang-Ming University (IACUC number: 1070410). All the experiments were conducted according to “Protocol for the Successful Induction of Collagen-Induced Arthritis (CIA) in Mice” (Chondrex, Redmond, Wash., USA).

Briefly, eight weeks old DBA/1J mice were injected intradermally (i.d) of 100 μg of bovine type 11 collagen (CII) (Chondrex, Redmond, Wash., USA) emulsified with Complete Freund's Adjuvant (CFA) (Chondrex, Redmond, Wash., USA) at the base of the tail. For a booster injection, Incomplete Freund's adjuvant (WA) containing 100 μg of CII was performed on the day 21. Arthritis would develop 28-35 days after the first injection.

The group of CIA mouse model comprises Sham: without collagen induction. RA: CIA mice without hUCMSCs administration, hUCMSCs: CIA mice with 1×10⁶ hUCMSCs administration, IL-1β-hUCMSCs: CIA mice with 1×10⁶ IL-1β pre-stimulated hUCMSCs.

On day 36, 1×10⁶ of hUCMSCs in 100 μl PBS were administered via tail vein. The severity of RA symptom was evaluated from the paw thickness with a digital caliper and the body weight was measured with an electronic scale every 10 days for four periods. The animal positron emission tomography-computerized tomography ([¹⁸F]FDG/micro-PET-CT) was used to evaluate the effect of IL-1β-hUCMSC on arthritis on day 76 after starting CIA induction with CII (that is day 40 after hUCMSCs administration) and mice were sacrificed to examine the degree of bone erosion and synovial hyperplasia using H&E staining.

According to the “Mouse Arthritis Scoring System” (Chondrex, Inc), clinical score was judged on the redness and swelling of the front and hind paws of each mouse. Three joint types are observed for scoring: the interphalangeal joint, the metacarpophalangeal joint, and the carpal and tarsal joint. The score is defined as the follow: score 0=normal, score 1=one joint type has redness and swelling, score 2=two joint types have redness and swelling, score 3=all three joints have redness and swelling, score 4=maximally severe both symptoms of the entire paw and hard to distinguish the anatomic appearance. The total score was obtained from 4 paws, so the maximum score was 16 for each mouse.

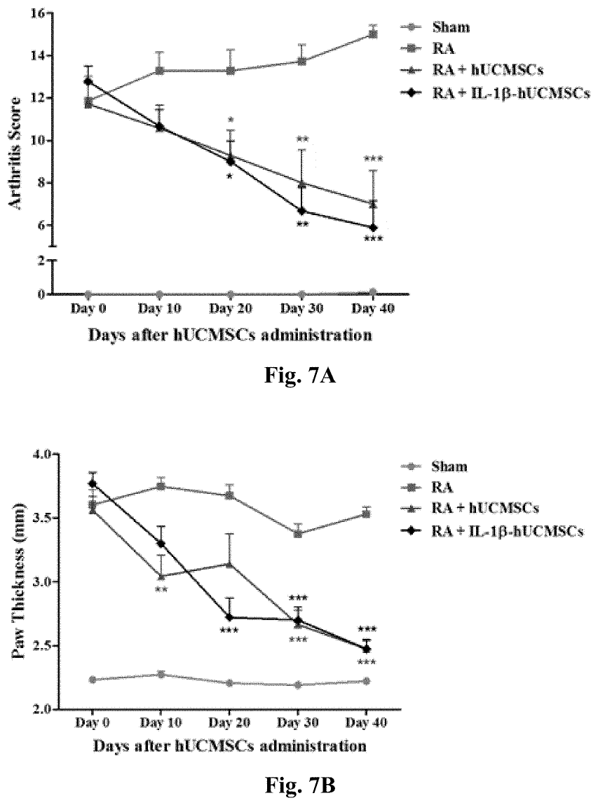

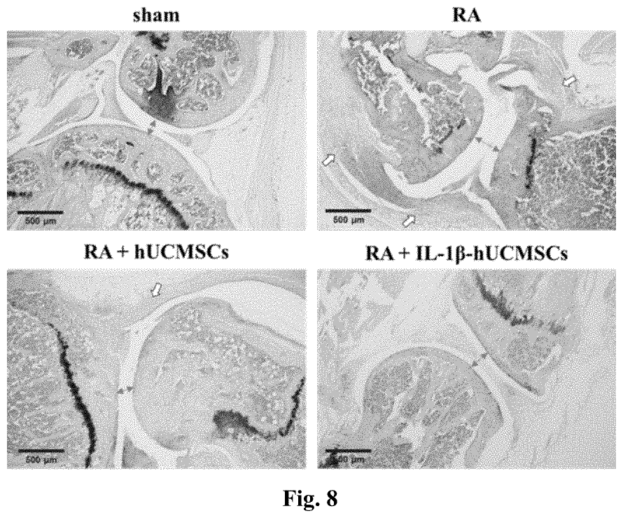

Therapeutic efficacy was also examined by photographing the exterior appearance of the front and hind paws. The mice in the RA group were shown with redness and severe swelling compared to the sham group. There was no significant change in body weight from day 0 to day 40 after hUCMSCs administration (FIG. 7A), The results of the arthritis score showed that the total score of both hUCMSCs and IL-1β-hUCMSCs were significantly decreased. However, the total score of the RA group gradually increased near the maximum value (FIG. 7B).

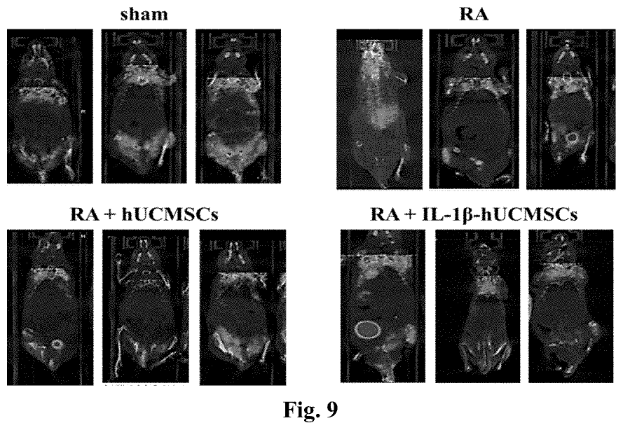

In order to confirm therapeutic efficacy, joint tissue was examined for pathological analysis by H&E staining. Mice were sacrificed on day 40 after hUCMSCs administration, the degree of bone erosion and synovial hyperplasia were examined. The results showed that less cartilage was preserved in the RA group, and that severe synovial hyperplasia was observed when compared to the sham group. The synovium was invaded to the bone area and the synovial joint was damaged. With hUCMSCs and IL-1β-hUCMSCs administration, more cartilage was preserved and synovial hyperplasia was inhibited (FIG. 8). Using [¹⁸F]FDG/microPET-CT to analyze the inflammation of arthritis, similar results were also obtained (FIG. 9).

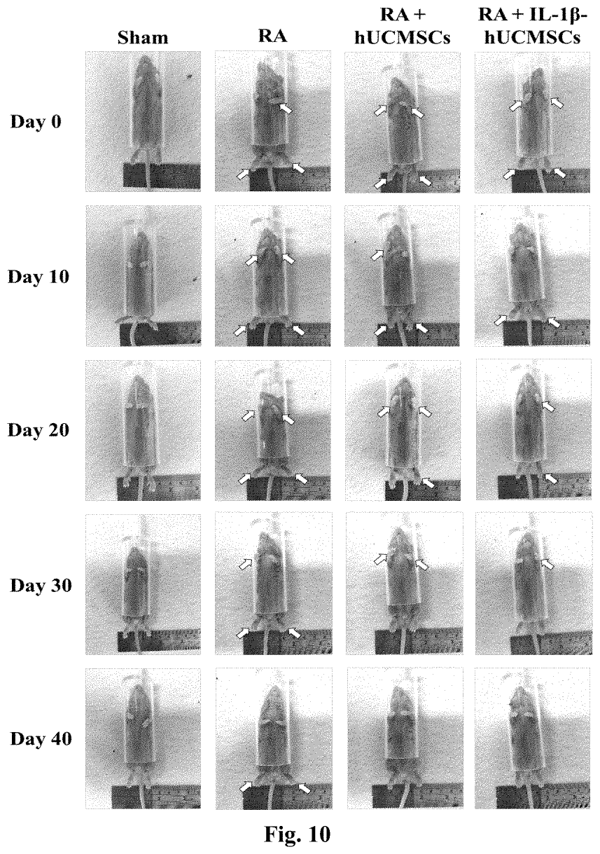

The therapeutic efficacy also examined by photographing their exterior appearance of the front and hind paws. The mice in the RA group were performed redness and severe swelling compared to the sham group. However, the RA symptoms were improved with hUCMSCs and IL-lii-hUCMSCs administration on day 40 compared to the RA group (FIG. 10).

The results of in vivo experiments demonstrate that IL-1β-stimulated hUCMSCs significantly alleviates the inflammation and swelling of rheumatoid arthritis, and diminishes bone erosion by synoviocytes. In the present disclosure, hUCMSCs were stimulated by cytokines (especially IL-1β) to: 1. promote the transendothelial migration of the hUCMSCs to the synovial inflammation sites; 2. promote adhesion to FLSs through the interaction of LEA-1/ICAM-1; and 3. initiate apoptosis induced by TRAIL.

1. A pharmaceutical composition for the treatment of rheumatoid arthritis, which is characterized by comprising interleukin-stimulated human umbilical cord mesenchymal stem cells (hUCMSCs) and pharmaceutically acceptable carriers, diluents or excipients.

2. The pharmaceutical composition of claim 1, wherein the interleukin-stimulated hUCMSCs are stimulated with interleukin for 8-72 hours.

3. The pharmaceutical composition of claim 2, wherein the interleukin-stimulated hUCMSC is stimulated with 5-1000 ng/ml interleukin for 16-48 hours.

4. The pharmaceutical composition of claim 1, wherein the interleukin is interleukin-1β.

5. The pharmaceutical composition of claim 1, wherein the interleukin-stimulated hUCMSC induces the apoptosis of human fibroblast-like synoviocyte in rheumatoid arthritis.

6. The pharmaceutical composition of claim 1, wherein the interleukin-stimulated hUCMSC inhibits synovial hyperplasia.

7. The pharmaceutical composition of claim 1, wherein the interleukin-stimulated hUCMSC diminishes bone erosion by synoviocytes.

8. The pharmaceutical composition of claim 1, wherein the interleukin-stimulated hUCMSCs alleviate inflammation and swelling of rheumatoid arthritis.

9. A use of interleukin-stimulated hUCMSCs in the manufacture of a medicament for treating rheumatoid arthritis, which is characterized in that the interleukin-stimulated hUCMSCs are stimulated with interleukin for 8-72 hours.

10. The use of claim 9, wherein the interleukin-stimulated hUCMSCs are stimulated with 5-1000 ng/ml interleukin for 16-48 hours.

11. The use of claim 9, wherein the interleukin is interleukin-1β.

12. The use of claim 9, wherein the interleukin-stimulated hUCMSC inhibits synovial hyperplasia.

13. The use of claim 9, wherein the interleukin-stimulated hUCMSCs alleviate inflammation and swelling of rheumatoid arthritis.

14. The use of claim 9, wherein the interleukin-stimulated hUCMSCs diminishes bone erosion in patients with arthritis.

15. A method for the treatment of rheumatoid arthritis, characterized in that the pharmaceutical composition of claim 1 is administered to patients in need.

16. The method of claim 15, wherein the interleukin-stimulated hUCMSCs are stimulated with interleukin for 8-72 hours.

17. The method of claim 16, wherein the interleukin-stimulated hUCMSCs are stimulated with 5-1000 ng/ml interleukin for 16-48 hours.

18. The method of claim 17, wherein the interleukin is interleukin-1β.
